(19) United States
(12) Patent Application Publication
(10) Pub. No.: US 2015/0073343 A1
(43) Pub. Date: Mar. 12, 2015

(54) CATHETER AND METHOD OF INSERTION

(71) Applicant: Iyunni Venkata Sesha Sayi Nath, Seminole, FL (US)

(72) Inventor: Iyunni Venkata Sesha Sayi Nath, Seminole, FL (US)

(21) Appl. No.: 14/492,895

(22) Filed: Sep. 22, 2014

Related U.S. Application Data
(63) Continuation of application No. 12/905,522, filed on Oct. 15, 2010, now abandoned.
Application No. 12/905,522 is a continuation of application No. 12/393,717, filed on Feb. 26, 2009, now Pat. No. 8,057,429.
(60) Provisional application No. 61/031,442, filed on Feb. 26, 2008.
(60) Provisional application No. 61/113,697, filed on Nov. 12, 2008.

(51) Int. Cl. A61M 29/00; A61M 25/10

(52) U.S. Cl. CPC A61M 29/00; A61M 25/1025
USPC 604/103.11

(57) ABSTRACT

A balloon catheter system includes a tube and a dilator detachable from the tube after the tube is inserted into a cavity in the patient. The cavity may be filled with gas or liquid to expand the viscera surrounding the cavity and the guidewire may be inserted using a trochar, for example. The tube includes one or more lumen to expandable balloons that are inflatable by fluid inserted through the lumen and into the balloon or balloons, which are formed by elastic film sealed on opposite ends to the external diameter of the tube. The dilator is held in place during insertion, such as by a stainless steel shaft and a sheath portion of the dilator extending over the end of the tube being inserted into the cavity. A portion of the sheath may extend over and protect the balloon. A retractor net may be used to capture and remove the dilator or the dilator may be designed as a bioabsorbable or digestible material or of a material that passes safely through the digestive tract of a patient.

### BACKGROUND

This application is a continuation of U.S. patent application Ser. No. 12/905,522 filed Oct. 15, 2010 which claims the benefit of and priority to U.S. patent application Ser. No. 61/031,442, filed Feb. 26, 2008, and U.S. patent application Ser. No. 61/113,697, filed Nov. 12, 2008, and U.S. patent application Ser. No. 12/393,717, filed Feb. 26, 2009 the Applicant incorporates by reference herein the entire specification of both of applications, and is incorporated herein by reference in its entirety (the “Nath Application”). The state of the art does not disclose a catheter for use in a percutaneous, minimally invasive procedure that utilizes a detachable dilator. Also, the existing devices are not capable of being used effectively for procedures as varied as gastric feeding, gastric decompression, jejunal feeding, cystostomy, nephrostomy, weight loss and colostomy.

### FIELD OF THE INVENTION

The field relates to a catheter for insertion into a patient using an endoscopic percutaneous insertion method or minimally invasive procedures.

### SUMMARY

The examples of the invention comprise a balloon catheter for use in a wide variety of procedures including, without limitation, one or more of the following procedures: gastric feeding, gastric decompression, jejunal feeding, cystostomy, bladder decompression, colonoscopy, nephrostomy and weight loss. In one example, a single balloon device may be used for all of the following indications for use: gastric feeding, gastric decompression, cystostomy, bladder decompression, nephrostomy and colostomy. In another example, a double balloon device may be used for one or more of these and for weight loss. In yet another example, a device including a plurality of balloons may be used for one or more of the following: gastric feeding, gastric decompression, jejunal feeding and weight loss.

For example, a catheter includes a balloon inflatable using a fluid, such as water or saline, which is capable of acting to retain the catheter in a cavity or lumen, such as the stomach, bladder or colon, for example. When inflated, the balloon is pulled against the wall of the lumen and seals the lumen. The catheter may be inserted under observation using minimally invasive techniques, such as an endoscope. The procedure is modified from the process disclosed in the Nath Application. Instead, a detachable dilator is arranged on the end of the catheter such that an introducer needle, shaft or rod extends through the detachable dilator. The dialer may be detached and retrieved using a snare, which may be used to remove the dilator through an endoscopic passage or other minimally invasive method. Alternatively, the detachable dilator may be made of a material that is capable of being bioabsorbed or may be capable of being detached from the catheter and passing without adverse consequences. For example, certain starch-based materials and gels are capable of being bioabsorbed and/or passing without adverse consequences. Also, it is believed, without being limiting in any way, that a silicone rubber dilator would be capable of passing through the digestive tract without adverse consequences. Other materials for the dilator tip may be a PEEK or a polyethylene, such as high density polyethylene. PEEK is a polyether ether ketone, which may be colorless, and is a known biocompatible engineering thermoplastic material.

The device and procedure provides advantages over any known method for inserting a comparatively large catheter, which may be any type of tube, into a cavity or lumen in a patient, which may be a human patient or a non-human patient, such as an animal Insertion of the catheter is greatly simplified using the detachable dilator. A guide wire, preferably a soft tip guide wire, is inserted in a location where catheterization is desired, using a trochar or other device as is known in the art The end of the guide wire opposite of the end inserted into the patient is inserted into an annular shaft that extends through the catheter and at least a portion of the dilator. The annular shaft, dilator and catheter tube may be pre-assembled. The assembly may be guided along the guidewire and into the patient, with or without lubrication of the catheter.

For example, the annular shaft may be an introducer needle, which may be made of any stiff, biocompatible material, such as a metal. In one example, the metal is stainless steel. The introducer needle is used only during insertion and is removed after the catheter is properly positioned within the patient.

The dilator follows the guidewire into the patient, widening the entrance by its transition from about the same diameter as the guide wire or the annular shaft to a diameter about the same diameter or larger than the outer diameter of the catheter. For example, the shape of the nose of the dilator may be a cone, truncated cone or paraboloid. A substantially conical outer surface of a dilator nose may be used, for example. Alternatively, the outer surface of the dilator nose may have a substantially parabolic shape. One advantage of the use of a detachable dilator is that the dilator may have an integrated sheath protecting a balloon of a balloon catheter during insertion of the balloon catheter. Another advantage of a detachable dilator is that the dilator is not withdrawn through the catheter, itself. Therefore, the dilator may have an outer diameter larger than the inner diameter of the tubular device being inserted into a patient However, unlike external dilators, the catheter need not be inserted through the dilator. Instead, the nose of the dilator may be sized to be the same external diameter as the external diameter of the catheter, because the dilator is dislodged from the catheter after the catheter is properly positioned in the patient Surprisingly, the detachable dilator reduces the time to perform an insertion procedure while avoiding, or at least reducing, leakage from the cavity around the point of insertion during an insertion procedure.

Surprisingly, a colostomy procedure may use a balloon catheter with a detachable dilator to quickly and efficiently decompress, cleanse and treat a patient that would otherwise be faced with a colectomy. The colostomy uses the minimally invasive surgical techniques similar to a gastrostomy or cystostomy to introduce a balloon catheter in the colon. The balloon catheter may be used for decompression and drainage by connecting the catheter with a colostomy bag. In addition, access to the colon is provided using the catheter for treating an infection or the like. It is surprising and unexpected that essentially the same procedure may be used in not only colostomy but also for nephrostomy, cystostomy, gastrostomy and weight loss. The additional steps required for jejunostomy are the same as required for any jejunostomy procedure. The insertion procedure has fewer complications than known insertion procedures, requires fewer devices to be stocked and improves proficiency by providing a device capable of being used in a plurality of “ostomy’ procedures.

A double balloon catheter may be inserted into a patient's stomach in a gastrostomy procedure and may be used for weight loss. By inflating the two balloons, hunger is alleviated, helping the patient to lose weight and to gradually reduce the size of the stomach, while avoiding any blockage of nutrition and hydration. In one example, supplemental nutrition and/or hydration may be provided by gastric feeding, as prescribed by a physician. The dumbbell-shaped double balloon provides a channel for passage of nutrition and hydration through the stomach, while occupying space in the stomach, alleviating discomfort from an empty feeling in the stomach.

In one example, a balloon catheter system for insertion into a patient comprises a balloon catheter, an annular shaft and a detachable dilator. The balloon catheter may be comprised of a tube having a first end, a second end opposite of the first end, a central bore fluidically coupling an inlet at the first end of the tube with an outlet at the second end of the tube and a lumen fluidically coupling a connector disposed adjacent the first end of the tube with a balloon disposed adjacent to the second end of the tube, the balloon being formed by a tubular film sealed at opposite ends of the tubular film to an external surface of the tube. The lumen has a lumen outlet such that fluid injected into the connector and through the lumen exits through the external surface of the tube at the outlet and into an expandable cavity between the balloon, which is made of a flexible, resilient material, and the external surface of the tube. The balloon is capable of being expanded by injecting fluid into the expandable cavity through the lumen.

The detachable dilator may be coupled to the outlet of the tube, when assembled, such that the dilator is held in place during insertion of the tube. After the tube is inserted, the dilator is detached from the tube. The dilator may comprise a sheath portion and a nose, the nose having an external surface that transitions from a first external diameter at a nose tip to a second external diameter at a portion of the nose adjacent to the outlet of tube, when the dilator is coupled to the outlet of the tube. An internal surface of the nose provides the channel through which the annular shaft may be inserted. The sheath portion of the dilator may be formed of a flexible, resilient material, and may be formed in a shape such that it closes in on itself, when the dilator is detached from the tube. The entire dilator may be made of a digestible material and/or bioabsorbable material.

The annular shaft may extend entirely through the nose or may extend through only a portion of the nose of the dilator. For example, the annular shaft may butt up against a portion of the nose of the dilator at a constriction formed within the inner surface of the nose, and the dilator may be detachable from the tube using only the annular shaft, such as by pushing the annular shaft to force the dilator off of the end of the catheter tube. In one example, the annular shaft is attached to a control mechanism, allowing the annular shaft, which extends only partially through the nose, to be used to push the detachable dilator off of the catheter tube, after insertion of the catheter tube into the patient, when the control mechanism is activated. The annular shaft extends through the central bore of the tube and is capable of being removed from the central bore of the tube, after the tube is inserted into the patient, by pulling on an extraction portion of the annular shaft. The extraction portion may extend from the inlet of the tube or may be attached to an extractor that extends from the inlet of the tube. The annular shaft may be made of a stainless steel, for example. In one example, the annular shaft has a pointed tip extending from the nose of the dilator, when the dilator is coupled to the outlet of the tube.

The balloon catheter system may comprise a separate detachment mechanism in addition to the catheter tube, annular shaft and dilator. For example, the detachment mechanism may comprise a tubular annulus having an inner bore diameter fitting over the outer diameter of the annular shaft and a distal end capable of butting up against the nose of the dilator. By pushing the tubular annulus the detachment mechanism is capable of detaching the dilator from the tube of the catheter, for example. The detachment mechanism may include a control mechanism at a proximal end of the tubular annulus of the detachment mechanism and may include a locking mechanism, which positively locks the tubular annulus until the locking mechanism is unlocked, preventing the distal end of the tubular annulus from pushing the dilator. For example, the locking mechanism may prevent the tubular annulus from being rotated about the longitudinal axis of the tubular annulus. If rotated, about its axis, a guide may be aligned to engage channel allowing the tubular annulus to engage the nose of the dilator, pushing the dilator from the tube of the catheter.

A method of assembling the balloon catheter system may comprise insetting the annular shaft through the central bore of the tube and through at least a portion of the channel of the nose, and coupling the dilator to the outlet of the tube such that the sheath portion of the dilator extends over the outlet of the tube and over at least a portion of the balloon. If the sheath portion of the dilator is formed of a flexible, resilient material, which is closes in on itself, then prior to coupling the dilator to the outlet of the tube, the sheath portion may be unfolded and spread to allow insertion of the outlet portion of the tube into the sheath portion of the dilator.

A method of using the balloon catheter system may comprise selecting a pre-assembled balloon catheter system, threading a guide wire through the annular shaft, inserting the dilator, the tube and the annular shaft along the guide wire and into a cavity within the patient, until the balloon is within the cavity, and detaching the dilator from the tube. The annular shaft may be extracted from the tube before or after the dilator is detached. The balloon of the catheter is expanded by injecting fluid into the expandable cavity between the balloon and the external surface of the tube. A retractable net may be inserted into the cavity of the patient to capture the dilator. Then the net and the dilator may be removed from the cavity of the patient, such as through a trochar or endoscope, used for minimally invasive surgical procedures. A control mechanism may be used to control the detachment of the dilator and the control steps may include unlocking the control mechanism, withdrawing the control mechanism beyond a detent in the control mechanism, and rotating the control mechanism about an axis of the control mechanism, prior to pushing the dilator away from the outlet of the tube, using the tubular annulus or the annular shaft or both as a push rod, for example.

### BRIEF DESCRIPTION OF THE DRAWINGS

FIGS. 1A-1C illustrate a detachable dilator as seen (A) externally and (B) in a cross sectional view, while the sheath portion is spread for insertion of the catheter tube, and (C) in a cross sectional view with the sheath portion folded in on itself.

FIG. 2 illustrates an example of the use of a detachable dilator, shown in cross section, fitted with a needle introducer and a balloon catheter.

FIG. 3 illustrates another example of the use of a detachable dilator, shown in cross section, fitted with a needle introducer, a balloon catheter, in partial cross section, and an example of a detacher.

FIG. 4 illustrates a perspective view of an example of a detacher head.

FIG. 5 illustrates a perspective view of an example of a detacher handle.

FIG. 6 illustrates a partially exploded view of a detacher head, a detacher handle and a needle introducer.

FIG. 6A illustrates a perspective view of an example of a double balloon feeding tube with both balloons expanded; FIG. 6B illustrates a cross sectional view of the feeding tube of FIG. 6A.

FIG. 7 illustrates a detail, perspective view of a balloon separate from the feeding tube.

### DETAILED DESCRIPTION

The examples described and the drawings rendered ate illustrative and are not to be read as limiting the scope of the invention as it is defined by the appended claims.

The method of insertion of a balloon catheter using a detachable dilator is similar to the procedure used in the Nath Application. However, instead of withdrawing the needle with a flexible sheath through the large channel in the feeding tube of the Nath Application, only the needle introducer is withdrawn through the catheter. The dilator is detached from the catheter after insertion of the catheter. In one example, the dilator is made of a biocompatible and/or digestible polymer. For example, a polytetrafluoroethylene may be used for the dilator and provides exceptional biocompatibility together with advantageous tribology. However, other polymer materials may be used, such as polyvinylchloride, polyethylene, polypropylene, polyethylene terephthalate, other polyesters, polymethylmethacrylate, polycarbonates, polyamides, polyacetals, polysulfones and co-polymers, such as tetrafluoroethylene and hexafluoropropylene and polyurethane block co-polymers.

In one example, the dilator is withdrawn using minimally invasive techniques. For example, a snare, such as one of the net-like snares capable of being used with an endoscope, is used to capture and remove the dilator. For example, a Roth Net® foreign body and rigidity, but hydrolyzing breaks the polylactide down to lactic acid, which digestible or passes harmlessly through the digestive tract. Other bioabsorbable materials include poly(trimethyl carbonate), poly(carbonate) and starch-based polymers and gels, which may provide sufficient rigidity to serve as a dilator. ¹ ROTH NET® is a trademark of US Endoscopy.

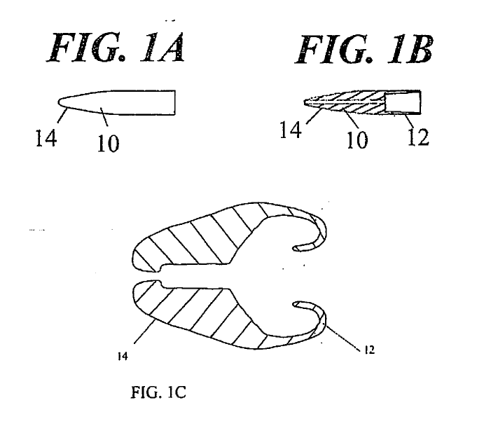

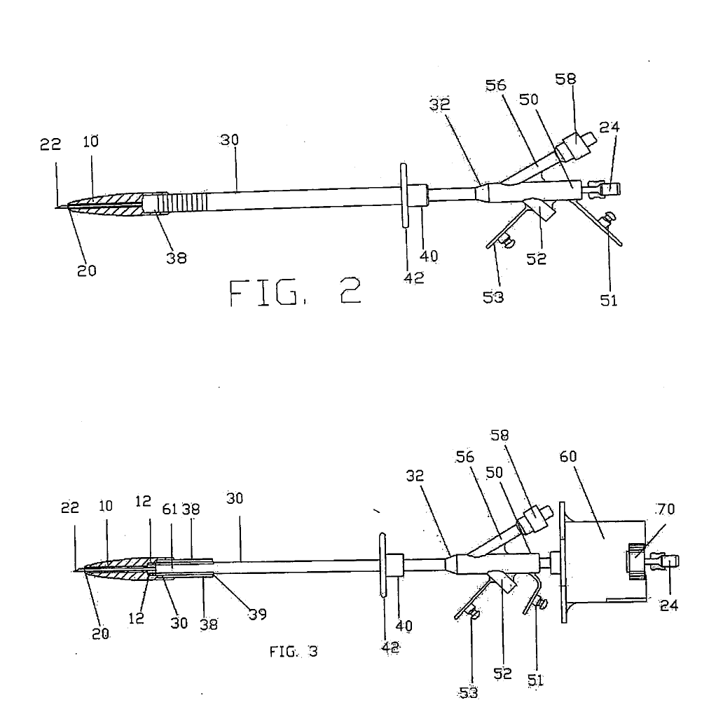

FIG. 1A illustrates a dilator 10, having an asymmetric nose 14 and an integrally formed protective sheath 12, as illustrated in the cross sectional view of FIG. 1B. In FIG. 1C, a resilient, flexible material, such as PEEK or silicone rubber, is used to form the sheath portion of the dilator, and the sheath portion is capable of returning to a shape folded in on itself. This may make it possible for the dilator to pass through the digestive tract with no harm to the patient, for example, and reduces the length of the dilator, if it is to be captured by a net, for example. FIG. 2 illustrates use of the detachable dilator 10 with a needle introducer 20 and a tri-funnel balloon catheter 30. The needle introducer is inserted through a channel defined by the inner diameter of the annular dilator 10. In one example, the needle introducer 20 and dilator 10 are sized, such that the needle introducer may be withdrawn from the dilator 10 and the catheter 30 by pulling the protector 24 while pushing on the catheter 30 to keep the catheter 30 stationary. In this way, both the needle introducer 20 and a guide wire inserted through the needle introducer may be withdrawn from the catheter 30. The dilator 10 is then free to be detached from the catheter 30. In one example, an integrally formed sheath 12 is thin, such as less than 1 millimeter in thickness, and non-rigid, and the dilator 10 dislodges from the catheter 30 as soon as the needle introducer 10 is removed. Alternatively, the dilator 10 may be dislodged from the catheter 30 by expanding the balloon 38, which is attached at the end of the catheter 30 by adhesive bonds 39 on each end of the balloon, for example. A balloon lumen 56 may be coextruded such that fluid inserted by syringe at the luer lock 58 inflates the balloon with a liquid, such as water or saline. A plurality of balloons may be provided by having additional coextruded balloon lumen, for example. The sheath 12 may protect each of the plurality of balloons, a single balloon or a portion of a single balloon. FIG. 2 illustrates a sheath protecting a leading edge of a balloon 38, for example.

An external bolster 40 provides a flange 42 that may be slid downward, once the balloon 38 is inflated and the catheter 30 is in place. In the example of FIGS. 2 and 3, a tri-funnel connector 50 is attached to the tube of the catheter by a transition region 32. The catheter has a main feeding channel, which may be closed by a flexibly attached plug 51 and a secondary drainage port 52, which may be connected to a drainage bag. The main channel may be flushed with sterile water or saline to keep the catheter free of obstructions and dots. A second plug 53 may be used to close the drainage port 52, when it is not being used. A luer lock 58 seals the balloon inflation lumen 56, which may be used to fill or drain the balloon 38 using a syringe.

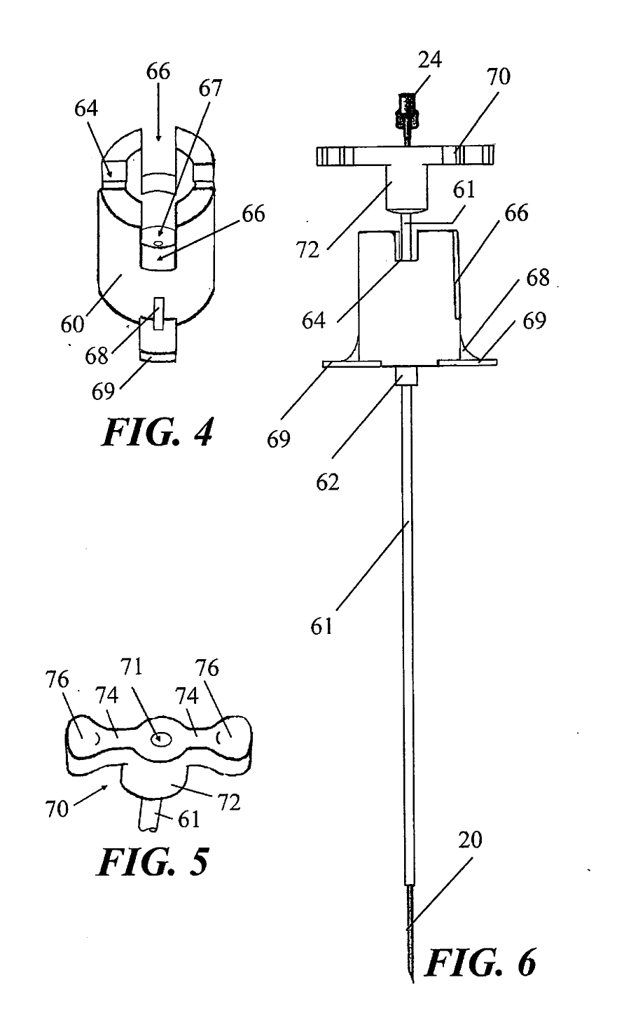

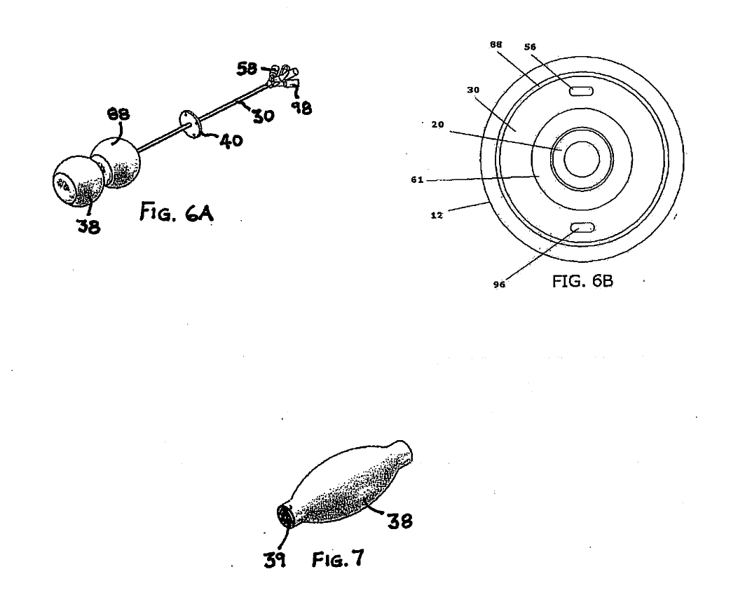

FIG. 3 illustrates the use of a detachment mechanism 60, 70, which includes a head 60, a handle 70 and a hollow push rod 61, which has a channel through which the needle introducer 20 extends, when preassembled prior to insertion, for example. As illustrated in FIG. 4, the head 60 has finger holds 69 on one end and slots or channels 64, 66 on the opposite end. The slots 64, 66 are dimensioned to accommodate the narrow portions 74 of the handle 70. A central bore 67 of the head 60 is sized to accommodate the diameter of the push rod 61. The push rod 61 is coupled to the handle 70 and one end of the push rod 61 extends from one side of the handle 70. The opposite end of the push rod 61 is capable of contacting the dilator 10 to force detachment of the dilator 10 from the catheter 30, when the handle 70 is backed-off, and rotated 90 degrees in the head 60, as illustrated in FIG. 6, aligning the narrow portion 74 of the handle 70 with the elongated channels 66. The user's fingers may compress the finger holds 76 of the handle 70 toward the finger holds 69 of the head 60, which are reinforced by arcuate supports 68. The head 60 has a funnel shape surrounding the central bore 67, which provides a centering function when the push rod 61 is inserted into the central bore 67. A tapered retention plug 62 extends from the tubeward side of the head 60 and is capable of mating with the main channel of the tri-funnel connector 50 as illustrated in FIG. 3. A slightly tapered plunger barrel 72 extends into the head 60 and acts to guide the handle 70 into the head 60 during compression. The handle 70 includes a central bore 71 that is capable of accommodating the needle introducer 20, as illustrated in FIG. 6. The protector 24 on the outward end of the needle introducer 20 may be held with one hand, while the other hand is used to plunge the handle into the head, keeping the needle introducer 20 in place while the dilator 10 is detached from the catheter 30. Alternatively, the needle introducer may be withdrawn prior to detaching the dilator 10 using the detachment mechanism 60, 70.

In FIG. 6A-6B, a double balloon (dumbbell-shaped) catheter is illustrated. The addition of a second balloon 88, in addition to the balloon nearest the outlet of the catheter 38, is provided to use in patients where a single balloon is insufficient. One or both of the balloons may have a larger volume than a single balloon device. For example, if a single balloon device has a balloon is filled with 20 cubic centimeters of liquid, and then one or both of the double balloons may be filled with 40 cubic centimeters of liquid. Alternatively, each balloon may be filled with the same volume of liquid, for example 20 cubic centimeters. In one example, each of the balloons in the double balloon device are capable of being filled to a maximum volume of 120 cubic centimeters, and use of the catheter is indicated for obese patients that have failed to control weight through diet and exercise. Each balloon has its own, independent luer lock 58, 59 for expansion and contraction of the balloon volumes in FIGS. 6A-6B. The gap between the two balloons 38,88 provides a path for food and liquid to pass from the esophagus, through the stomach and to the intestines. A feeding port and drainage port are provided as illustrated in the single balloon catheter of FIGS. 2-3.

A typical balloon 38 is illustrated in FIG. 7, and the adhesive layer 39 is represented by the cross-hatched region on the end of the balloon.

Additional examples and configurations of the features of the examples shall be understood based on these examples and are intended to be included within the scope of the claims, which are not to be limited by the details of the examples provided.

What is claimed is:

1. A balloon catheter system for insertion into a patient, using a guidewire previously inserted into a cavity of the patient, the system comprising:
a balloon catheter comprised of a tube having a first end, a second end opposite of the first end, a central bore fluidically coupling an inlet at the first end of the tube with an outlet at the second end of the tube and a lumen fluidically coupling a connector disposed adjacent the first end of the tube with a balloon disposed adjacent to the second end of the tube, the balloon being formed by a tubular film sealed at opposite ends of the tubular film to an external surface of the tube, the lumen having a lumen outlet such that fluid injected into the connector and through the lumen exits through the external surface of the tube at the lumen outlet and into an expandable cavity between the balloon, which is made of a flexible, resilient material, and the external surface of the tube, whereby the balloon is capable of being expanded by injecting fluid into the expandable cavity through the lumen;
a detachable dilator, comprised of a sheath portion extending from a solid body, the solid body having a central bore passing through the solid body such that an annular shaft passing through the central bore of the tube of the balloon catheter and the central bore of the solid body removably retains the detachable dilator on the annular shaft at the second end of the tube, and the sheath portion extends seamlessly from a portion of the solid body having a first diameter, the first diameter being sized to an outer diameter of the tube such that the sheath portion extends over the outer diameter of the tube, and the solid body has a tip distal from the portion of the solid body having a first diameter, the tip having a second diameter, and the solid body having a transition region having an external diameter decreasing over a length of the transition region from the second diameter, larger than the first diameter, to the first diameter at the tip of the detachable dilator, the first diameter at the tip being substantially the same as an outer diameter of the annular shaft, and the annular shaft having an inner diameter greater than the diameter of the guidewire, such that the guidewire is insertable into the inner diameter of the annular shaft, and the annular shaft is guidable over the guidewire, the detachable dilator extending into the cavity of the patient as the annular shaft is guided over the guidewire, the detachable dilator being slidably engaged on the annular shaft, such that, when the annular shaft is withdrawn from the central bore of the tube, an inner surface of the central bore of the solid body slides over an external surface of the annular shaft, releasing the detachable dilator to detach from the annular shaft when the annular shaft is withdrawn from the tube, after inserting the tube into the cavity of the patient.

2. The balloon catheter system of claim 1, wherein the annular shaft is of a stainless steel.

3. The balloon catheter system of claim 2, wherein the annular shaft has a pointed tip extending from the detachable dilator.

4. The balloon catheter system of claim 1, wherein the sheath portion of the detachable dilator is formed of a flexible, resilient materials, and the sheath portion closes in on itself, when the detachable dilator is detached from the tube.

5. The balloon catheter system of claim 1, wherein the dilator is made of a digestable material.

6. The balloon catheter system of claim 1, wherein the sheath portion is made of a digestable material.

7. The balloon catheter system of claim 1, wherein the external diameter of the transition region changes non-linearly with length.
